(19) United States
(12) Patent Application Publication
(10) Pub. No.: US 2025/0131586 A1
(43) Pub. Date: Apr. 24, 2025

(54) INFORMATION PROCESSING APPARATUS, RADIATION IMAGING SYSTEM, INFORMATION PROCESSING METHOD, AND COMPUTER-READABLE STORAGE MEDIUM

(71) Applicant: CANON KABUSHIKI KAISHA, Tokyo (JP)

(72) Inventor: Hideaki MIYAMOTO, Tokyo (JP)

(21) Appl. No.: 18/912,669

(22) Filed: Oct. 11, 2024

(30) Foreign Application Priority Data
Oct. 24, 2023 (JP) 2023-182678

(51) Int. Cl. G06T 7/73; G06V 10/25; G06V 10/44; G06V 10/764; G06V 10/771; G16H 30/40

(52) U.S. Cl. CPC G06T 7/73; G06V 10/25; G06V 10/44; G06V 10/764; G06V 10/771; G16H 30/40; G06T 2207/20044; G06T 2207/20081

(57) ABSTRACT

An information processing apparatus includes: an obtaining unit that obtains an optical image obtained by imaging an object in an imaging scene of a radiation image; an estimating unit that estimates skeletal information of the object in the obtained optical image by using the obtained optical image as input data of a second learned model, the second learned model obtained by performing incremental learning on a first learned model that estimates skeletal information relating to a skeletal structure of an object, the incremental learning performed by using skeletal information relating to a skeletal structure different from a skeletal structure indicated by skeletal information learned by the first learned model; and a determining unit that determines object information which includes information of at least one of laterality and a site of the object in the optical image by using the skeletal information of the object in the optical image.

### BACKGROUND OF THE INVENTION

### Field of the Invention

The present invention relates to an information processing apparatus, a radiation imaging system, an information processing method, and a computer-readable storage medium.

### Description of the Related Art

In recent years, in radiation imaging for a medical examination, imaging assist by an optical image has been performed, in which additional information obtained by optically imaging an imaging scene to obtain the optical image and analyzing the optical image is provided to the operator together with a live image. For example, Japanese Patent Application Laid-Open No. 2020-199163 proposed a technique for efficiently performing radiation imaging without depending on the skill or experience of an engineer by determining the imaging position of an object from an optical image and outputting information on the suitability of the imaging position.

In the technique disclosed in Japanese Patent Application Laid-Open No. 2020-199163, the imaging position of the object is determined from the optical image. However, in the image analysis technique applied to such imaging position determination, there is a problem of further continuous improvement of accuracy in a wider range of objects and imaging-conditions.

In general, in the image processing technique, it is known that learned model capable of highly accurate recognition and classification can be realized by a machine learning algorithm using a large amount of training data. However, it is difficult to collect a large amount of training data including the optical image, which is personal information of patients, from a radiation imaging scene for medical examination.

Therefore, in one embodiment of the present disclosure, it is an object to provide an information processing apparatus that can obtain object information from an optical image by using a learned model that estimates a desired skeletal information, which is obtained by using a reduced number of training data as compared with the conventional one.

### SUMMARY OF THE INVENTION

A information processing apparatus according to an embodiment of the present disclosure includes: an obtaining unit configured to obtain an optical image obtained by imaging an object in an imaging scene of a radiation image; an estimating unit configured to estimate skeletal information of the object in the obtained optical image by using the obtained optical image as input data of a second learned model, the second learned model obtained by performing incremental learning on a first learned model that estimates skeletal information relating to a skeletal structure of an object, the incremental learning performed by using skeletal information relating to a skeletal structure different from a skeletal structure indicated by skeletal information learned by the first learned model; and a determining unit configured to determine object information which includes information of at least one of laterality and a site of the object in the optical image by using the skeletal information of the object in the optical image.

Further features of the present invention will become apparent from the following description of exemplary embodiments with reference to the attached drawings.

### BRIEF DESCRIPTION OF THE DRAWINGS

FIG. 1 is a diagram for illustrating a schematic configuration of a radiation imaging system according to an embodiment.

FIG. 2 is a diagram for illustrating a schematic configuration of an information processing apparatus according to the embodiment.

FIG. 3 is a flowchart showing a processing procedure according to a first example.

FIG. 4 is a diagram for describing an example of an optical image according to the first example.

FIG. 5 is a diagram for describing an example of output of a skeletal structure estimating unit according to the first example.

FIG. 6 is a flowchart showing an example of object information determination processing according to the first example.

FIG. 7 is a diagram for describing arrangement of a radiation generating apparatus in a camera coordinate system.

FIG. 8 is a flowchart showing an example of processing using object information according to the first example.

FIG. 9 is a flowchart showing a processing procedure according to a second example.

FIG. 10 is a diagram for describing an example of an optical image according to the second example.

FIG. 11 is a diagram for describing an example of output of a skeletal structure estimating unit according to the second example.

FIG. 12 is a flowchart showing an example of object information determination processing according to the second example.

FIG. 13 is a diagram for describing another example of the optical image according to the second example.

FIG. 14 is a flowchart showing another example of the object information determination processing according to the second example.

FIG. 15 is a flowchart showing a processing procedure according to a third example.

### DESCRIPTION OF THE EMBODIMENTS

Hereinafter, an exemplary embodiment and exemplary examples for implementing the present disclosure will be described in detail with reference to the drawings. However, the dimensions, materials, shapes, relative positions, and the like of the components described in the following embodiment and examples can be freely set and may be modified according to the configuration of the apparatus to which the present disclosure is applied or various conditions. Further, the same reference numerals are used between the drawings to indicate elements that are identical or functionally similar in the drawings.

In the following description, the term “radiation” may include, for example, electromagnetic radiation such as X-rays and γ-rays, and particle radiation such as α-rays, β-rays, particle rays, proton rays, heavy ion rays, and meson rays.

In the following, the machine learning model refers to a learning model based on a machine learning algorithm. Specific algorithms of the machine learning include the nearest neighbor method, the naive Bayes methods, the decision trees, and the support vector machines. There specific algorithms include is a deep learning that generates characteristic amount and combining weighting factors for learning by itself using a neural network. As an algorithm using a decision tree, there is also a method using gradient boosting such as LightGBM and XGBoost. Appropriately available algorithms can be used in the following embodiments and example. The term “teacher data” refers to training data and is composed of a pair of input data and output data. The output data of training data is also referred to as ground truth.

Further, a learned model refers to a model that has been performed training (learning) on a machine learning model that accords to any machine learning algorithm such as the deep learning using appropriate teacher data (training data) in advance. The learned model has been obtained by training using the appropriate training data in advance, however the learned model is not a model which does not perform further training, and the incremental learning may be performed on the learned model. The incremental learning can be performed even after the apparatus is installed at the place of use.

### Embodiment

### (Schematic Configuration)

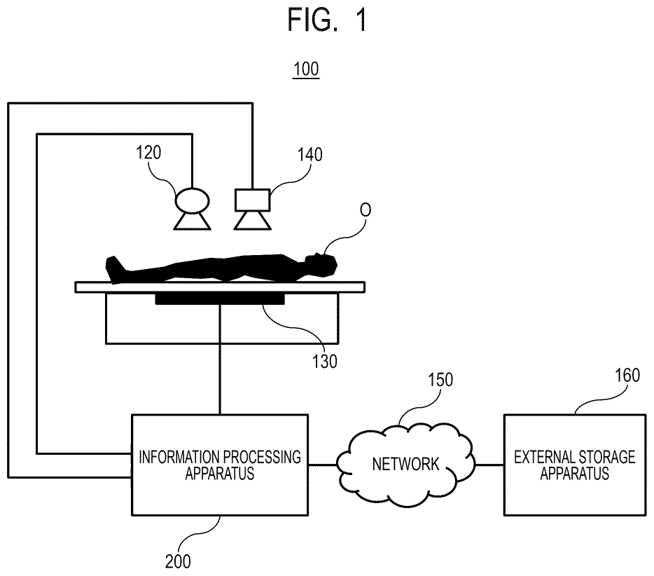

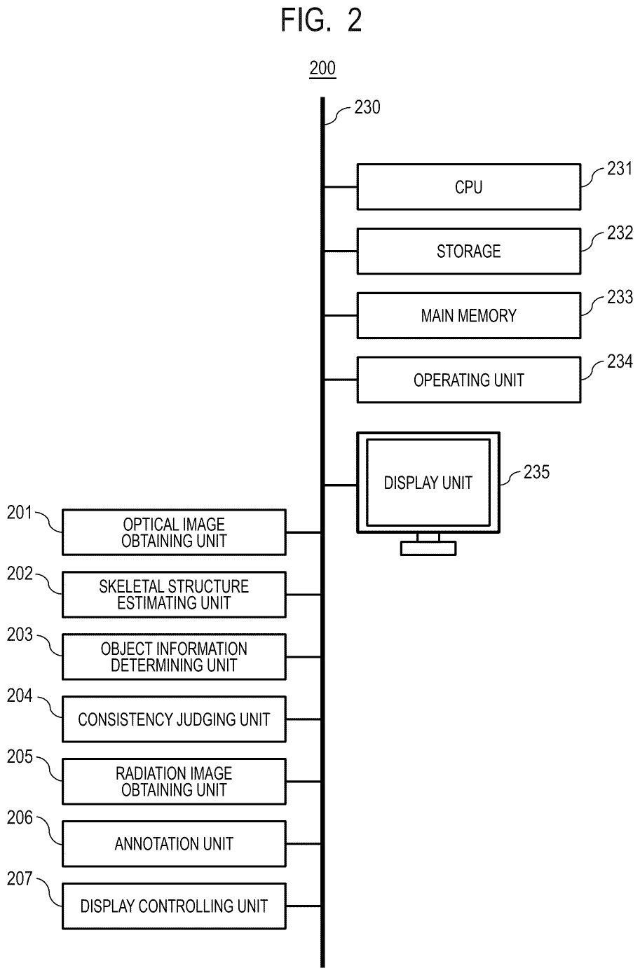

First, a radiation imaging system, an information processing apparatus, and an information processing method according to an embodiment of the present disclosure will be described with reference to FIG. 1 and FIG. 2. An embodiment of the present disclosure is applied to, for example, a radiation imaging system 100 and an information processing apparatus 200 shown in FIG. 1 and FIG. 2. FIG. 1 is a diagram for illustrating a schematic configuration of the radiation imaging system 100 according to the embodiment of the present disclosure, and FIG. 2 is a diagram for illustrating a schematic configuration of the information processing apparatus 200 according to the embodiment of the present disclosure. An object O is shown in a recumbent position in FIG. 1, however the object O may be in, for example, a standing position or a sitting position. The imaging table used to support the object O may be a table corresponding to the position of the object O.

The radiation imaging system 100 includes the information processing apparatus 200, a radiation generating apparatus 120, a radiation detector 130, and a camera 140. The information processing apparatus 200 is connected to the radiation generating apparatus 120, the radiation detector 130, and the camera 140 and can control them. Further, the information processing apparatus 200 can perform image processing and analysis processing of various images obtained using the radiation detector 130 and the camera 140. The information processing apparatus 200 is connected to an external storage apparatus 160 such as a server via any network 150 such as the Internet or an intranet, and can exchange data with the external storage apparatus 160. The external storage apparatus 160 may be directly connected to the information processing apparatus 200.

The radiation generating apparatus 120 includes, for example, a collimator, a collimator lamp, a radiation generator such as a radiation tube, and the like, and can irradiate a radiation beam under the control of the information processing apparatus 200. The radiation beam irradiated from the radiation generating apparatus 120 passes through the object O while attenuating, and enters the radiation detector 130.

The radiation detector 130 can detect the incident radiation beam and transmitting a signal corresponding to the detected radiation beam to the information processing apparatus 200. The radiation detector 130 may be any radiation detector that detects the radiation and outputs a corresponding signal, and may be configured using, for example, an FPD (Flat Panel Detector) or the like. The radiation detector 130 may be an indirect conversion type of detector that converts the radiation to visible light once using a scintillator or the like and converts the visible light to an electric signal using an optical sensor or the like, or a direct conversion type of detector that directly converts the incident radiation to an electric signal.

The camera 140 is an example of an optical apparatus that performs optical imaging of the object O under control of the information processing apparatus 200 and obtains an optical image. The camera 140 transmits the optical image obtained by the imaging to the information processing apparatus 200. The camera 140 may have any known configuration, and may be configured as a camera which can image a movie, such as a video camera, or as a camera that only takes still images. The camera 140 may be configured to take images using visible light, or may be configured to take images using invisible light other than radiation, such as infrared light.

The information processing apparatus 200 includes an optical image obtaining unit 201, a skeletal structure estimating unit 202, an object information determining unit 203, a consistency judging unit 204, a radiation image obtaining unit 205, an annotation unit 206, and a display controlling unit 207. The information processing apparatus 200 also includes a CPU231, a storage 232, a main memory 233, an operation unit 234, and a display unit 235. The respective units of the information processing apparatus 200 are connected via a CPU bus 230 and can exchange data with each other.

The optical image obtaining unit 201 controls the camera 140 and can obtain the optical image of object O imaged by the camera 140. The optical image obtaining unit 201 may obtain the optical image of the object O from the external storage apparatus 160, an optical apparatus (not shown) connected to the information processing apparatus 200 via any network, or the like. The optical image obtaining unit 201 may obtain the optical image stored in the storage 232.

The skeletal structure estimating unit 202 performs skeletal structure estimation using the optical image as input data of a learned model, and can estimate skeletal information of a human body in the optical image. The learned model used by the skeletal structure estimating unit 202 according to this embodiment may be a learned model generated by perform incremental learning using desired data on a general-purpose learned model for estimating skeletal information obtained by using many training data. Here, the desired data may include skeletal information or the like desired in a medical institution or a medical scene where the radiation imaging system 100 is used. The detailed processing of the skeletal structure estimating unit 202 will be described later.

The object information determining unit 203 analyzes the estimated skeletal information, and can determine and recognize object information including information indicating a site, information indicating laterality, information indicating direction, and the like of the object O which is an imaging-target of a radiation image in the optical image. The detailed processing of the object information determining unit 203 will be described later.

The consistency judging unit 204 can determine whether the object information determined by the object information determining unit 203 is consistent with the information regarding object O included in an imaging order of the radiation image. The information processing apparatus 200 can assist in determining whether the object information obtained using the optical image is consistent with an instruction of the radiation imaging by providing the determination result to the operator.

The radiation image obtaining unit 205 can control the radiation generating apparatus 120 and the radiation detector 130 to perform the radiation imaging of the object O, and obtain the radiation image of the object O from the radiation detector 130. The radiation image obtaining unit 205 may obtain the radiation image of the object O from the external storage apparatus 160 or a radiation detector (not shown) connected to the information processing apparatus 200 via any network. The radiation image obtaining unit 205 may obtain the radiation image stored in the storage 232.

The annotation unit 206 annotates the object information determined by the object information determining unit 203 to the radiation image. Here, the annotation refers to a process of embedding the object information indicating the site, the laterality, and the direction of the object O in the radiation image. The annotation unit 206 may be configured to annotate the object information to the optical image.

The display controlling unit 207 can control the display of the display unit 235. The display controlling unit 207 can cause the display unit 235 to display, for example, patient information about a patient as the object O, imaging-condition, parameters set by the operator, the generated optical image and radiation image, the determined object information, the analysis information, and the like. The analysis information may include, for example, segmentation information and the like. Further, the display controlling unit 207 can cause the display unit 235 to display any display such as a button or a slider for accepting an operation by the operator, a GUI or the like, in accordance with a desired configuration.

The CPU (central processing unit) 231 is an example of a processor for controlling the operation of the information processing apparatus 200. The CPU231 performs the control of the operation of the entire apparatus according to operations from the operation unit 234 and parameters stored in the storage 232 by using the main memory 233. The processor in the information processing apparatus 200 is not limited to the CPU and may include, for example, a microprocessing unit (MPU) and a graphics processing unit (GPU). The processor may also include a digital signal processor (DSP), a data flow processor (DFP), and a neural processing unit (NPU).

The storage 232 can store various images, data, and the like processed by the information processing apparatus 200. The storage 232 can store the patient information, the imaging-condition, the parameters set by the operator, and the like. The storage 232 can store information on a rule-based algorithm of the analysis for the skeletal information performed by the object information determining unit 203, and the like. The storage 232 may be configured by any storage medium such as an optical disk or memory. The main memory 233 is configured by a memory or the like, and can be used for temporary data storage or the like.

The GPU can perform efficient arithmetic operations by parallelly processing more data. Therefore, in a case of performing training a plurality of times using a machine learning algorithm such as deep learning, it is effective to perform the processing with the GPU. Therefore, in this embodiment, in the processing by the information processing apparatus 200 which functions as an example of a training unit, GPU may be used in addition to a CPU. Specifically, when training program including a learning model is executed, the CPU and the GPU cooperatively perform the arithmetic operations to perform learning. In the processing by the training unit, the arithmetic operations may be performed only by the CPU or the GPU. Further, the estimation processing according to this embodiment may also be implemented using a GPU as in the case of the training unit. In a case where the learned model is provided in an external device, the information processing apparatus 200 may not function as the training unit.

The training unit may include an error detecting unit and an updating unit (not shown). The error detecting unit obtains an error between the ground truth and output data output from the output layer of the neural network in accordance with input data input to the input layer. The error detecting unit may calculate the error between the output data from the neural network and the ground truth by using a loss function. The updating unit updates connection weighting factors or the like between nodes of the neural network based on the error obtained by the error detecting unit so that the error becomes small. The updating unit updates the connection weighting factors or the like using, for example, an error back-propagation method. The error back-propagation method is a method of adjusting the connection weighting factors or the like between the nodes of each neural network so that the error becomes small.

As the machine learning model according to this embodiment, for example, FCN (Fully Convolutional Network), SegNet or the like can be used. As a machine learning model for the object recognition, for example, RCNN (Region CNN), fastRCNN or fasterRCNN can be used. Further, YOLO (You Only Look Once) or SSD (Single Shot Detector or Single Shot MultiBox Detector) may be used as a machine learning model for performing the object recognition in units of regions.

The operation unit 234 includes input devices for operating the information processing apparatus 200, and includes, for example, a keyboard and a mouse. The operator can also input parameters related to the rule-based algorithm of the analysis processing using the skeletal information performed by the object information determining unit 203 through the operation unit 234.

The display unit 235 includes, for example, any display, and displays various information, such as the object information, and various images under the control of the display controlling unit 207. The display unit 235 may be, for example, a monitor of a console for operating the radiation generating apparatus 120 and the radiation imaging apparatus including the radiation detector 130. Further, the display unit 235 may be a sub-monitor installed at a position where the operator can observe the sub-monitor while assisting in the positioning of the object O, or a console monitor of a radiation irradiator. Furthermore, the display unit 235 may be a display device by which the operator can reliably check the display with a small amount of eye movement, such as a head-mounted display on which the operator can work while wearing. The display unit 235 may be configured by a touch-panel display, and in this case, the display unit 235 can also be used as the operation unit 234.

The information processing apparatus 200 can be configured by a computer including a processor and a memory. The information processing apparatus 200 can be configured by a general computer or a computer dedicated to the radiation imaging system. For example, a personal computer (PC), and a desktop PC, a notebook PC, a tablet PC (portable information terminal), or the like can be used as the information processing apparatus 200. Further, the information processing apparatus 200 can be configured as a cloud type computer in which some components are arranged in an external device.

The optical image obtaining unit 201, the skeletal structure estimating unit 202, the object information determining unit 203, the consistency judging unit 204, the radiation image obtaining unit 205, the annotation unit 206, and the display controlling unit 207 can be configured by a software module executed by the CPU231. Further, each of these components can be configured by a circuit performing a specific function such as an ASIC, an independent apparatus, or the like.

Next, the operation of the information processing apparatus 200 under the control of the CPU231 will be described. First, the information processing apparatus 200 starts imaging preparation based on imaging order information transmitted from an information management apparatus (not shown), and the optical image obtaining unit 201 starts optical image obtainment under the control of the CPU231. The imaging order information is information corresponding to a unit of examination ordered by a physician, and includes, for example, the patient information, an imaging (scheduled) date and time, and information including a site, a direction, an attitude and the like of the imaging-target based on physician's observation. The imaging order information includes, for example, a kind (for standing, lying, portable, etc.) of the radiation detection apparatus to be used, a patient's attitude (imaged site, and directions, etc.), and the radiation imaging conditions (tube voltage, tube current, presence or absence of grid, etc.) as information necessary for the radiation imaging.

The optical image obtaining unit 201 controls the camera 140 to perform the imaging of the optical image of the object O, and obtains the optical image from the camera 140. The optical image obtained by the optical image obtaining unit 201 is sequentially transferred to the main memory 233, the skeletal structure estimating unit 202, and the object information determining unit 203 via the CPU bus 230.

The skeletal structure estimating unit 202 estimates the skeletal information of the object O by using the transferred optical image as the input data for the learned model. Subsequently, the object information determining unit 203 obtains the object information from the estimated skeletal information. The object information is transferred to the consistency judging unit 204 via the CPU bus 230. The consistency judging unit 204 compares the imaging order information with the object information and outputs a consistency determination result. The optical image, the skeletal information, the object information, and the consistency determination results are transferred to the storage 232 and the display controlling unit 207 via the CPU bus 230. The storage 232 stores the transferred various information. The display controlling unit 207 causes the display unit 235 to display the transferred various information.

The operator confirms the displayed various information and performs an operation instruction via the operation unit 234 as needed. For example, if the consistency determination result is correct, the operator performs an imaging instruction of the radiation image via the operation unit 234. The imaging instruction is transmitted to the radiation image obtaining unit 205 by the CPU231.

Upon receiving the imaging instruction, the radiation image obtaining unit 205 controls the radiation generating apparatus 120 and the radiation detector 130 to perform the radiation imaging. In the radiation imaging, the radiation beam is irradiated from the radiation generating apparatus 120 toward the object O, and the radiation beam transmitted through the object O while attenuating is detected by the radiation detector 130. The radiation image obtaining unit 205 obtains a signal corresponding to the intensity of the radiation beam detected by the radiation detector 130 as the radiation image. Data of the radiation image is sequentially transferred to the main memory 233 and the annotation unit 206 via the CPU bus 230.

The annotation unit 206 annotates the object information stored in the storage 232 to the transferred radiation image. The annotated radiation image is transferred to the storage 232 and the display controlling unit 207 via the CPU bus 230. The storage 232 stores the transferred annotated radiation image. The display controlling unit 207 causes the display unit 235 to display the transferred annotated radiation image. The operator can confirm the displayed radiation image and perform an operation instruction as needed via the operation unit 234.

### First Example

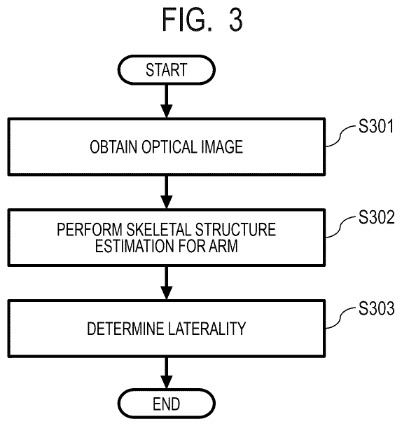

Hereinafter, a radiation imaging system, an information processing apparatus and an information processing method according to a first example of the present disclosure will be described with reference to FIG. 3 to FIG. 7. In the first example, a process of recognizing the laterality of the object to be imaged from an optical image obtained at a predetermined frame rate using a video camera as the camera 140, and outputting the laterality as the object information will be described. The laterality is information indicating whether a site to be imaged is left or right for a human site having left and right sides.

(Process Flow) A series of processing procedures according to the first example will be described below with reference to FIG. 3. FIG. 3 is a flowchart showing a processing procedure according to the first example. When the processing procedure according to the first example is started, the process proceeds to step S301.

### (Step S301)

In step S301, the optical image obtaining unit 201 controls the camera 140 to obtain an optical image of the radiation imaging scene including the object of the radiation imaging. In the first example, the camera 140 is a video camera mounted on the radiation generator, and images the object taking an imaging position on the radiation detector 130 arranged in a recumbent table, and outputs the optical images at a predetermined frame rate.

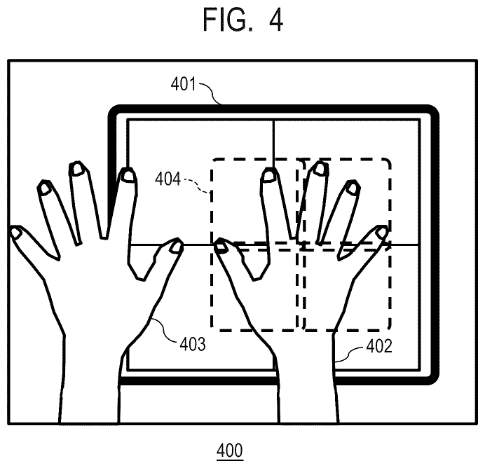

Here, an example in which the radiation detector 130, the right hand 402, and the left hand 403 appear in the optical image 400 will be described with reference to FIG. 4. FIG. 4 shows an example of the optical image according to the first example. A radiation detector region 401 in the optical image 400 is a region showing the radiation detector 130. Further, in the example shown in FIG. 4, it is assumed that light of a collimator lamp is irradiated on the right hand of the object, and the collimator lamp irradiation region 404 appears on the right hand 402 in the optical image 400. Here, the collimator lamp is an irradiating apparatus that produces visible light and is attached to a collimator for confirming a planned radiation irradiation region which is a region to be irradiated with the radiation before the radiation irradiation. The collimator lamp irradiation region 404 generated by the collimator lamp coincides with the planned radiation irradiation region at the time of radiation imaging.

### (Step S302)

In step S302, the skeletal structure estimating unit 202 estimates the skeletal information of the object by using the optical image obtained by the optical image obtaining unit 201 as the input data for the learned model. Here, the skeletal information is information representing a plurality of predefined feature points on the object, and includes coordinates representing a site such as, a face, a shoulder, a hand, a waist, and a foot, and can be used to recognize the position (posture) of a human body. In the first example, the skeletal structure estimating unit 202 uses a learned model comprised of a machine learning model represented by a neural network. Here, it is assumed that the learned model has performed the incremental learning using data corresponding to an output desired at the scene of radiation imaging, which had been generated by the supervised machine learning using various image data not limited to the scene of radiation imaging in advance.

First, in the head, the feature point may include, for example, at least one of eyes, a nose, and a mouth in order to distinguish the front and rear of the head. Also, in the torso, the feature point may include, for example, a feature point for distinguishing the neck, the chest (thoracic vertebrae), the abdomen, and the waist (lumbar vertebrae). Further, in the limbs, the feature point may include, for example, the shoulder, the elbow, the wrist, the hip joint, the knee, and an ankle. Since the skeletal structure estimating unit 202 estimates the skeletal information indicating these feature points, the information processing apparatus 200 can recognize a wide range of postures. In addition, a site having laterality, for example, can be defined as a right hand or a left hand, which are different feature point at the time of skeletal structure estimation. In this case, the information processing apparatus 200 can estimate the laterality based on the feature point.

While the skeletal structure estimation processing may be performed for the optical image using one neural network model, the skeletal structure estimation processing may also include the object region extraction processing for extracting the object region from the optical image and perform the skeletal structure estimation from the extracted object region. In this case, the object region in the optical image may be used as the input data of the learned model. The object region in the optical image may be used for training data. Further, the skeletal structure estimation processing may include the classification processing for classifying whether the whole body of the object or only a part such as a head, a hand, or a foot appears on the optical image. In this case, the skeletal structure estimating unit 202 may select and apply a learned model for the skeletal structure estimation for each class specialized for the whole body, the head, the arm, or the foot based on the result of the class classification processing.

In general, as the neural network model for performing the skeletal structure estimation, a learned model which is assumed to be used for general purposes and on which has been performed the machine learning using a large amount of data including an optical image and skeletal information of an object in the optical image is used. However, in a case where such a learned model is used as a skeletal structure estimating unit required in a medical scene which is a target of the first example, for example, feature points which can be output may lack. For example, regarding the limbs, it is assumed that the shoulder joint, the elbow joint, the wrist, the hip joint, the knee joint, and ankle are trained to be output, but the fingers, toes, and feature point representing the front and back of them cannot be output. Therefore, in the first example, a learned model obtained by performing the incremental learning on such a general-purpose learned model so that the feature point required for each medical institution or medical scene can be output is used. The incremental learning of the neural network model for additionally outputting such a feature point relating to a part of the human body site can be realized by machine learning using a relatively small data set compared with the data set used for the learning of the general-purpose learned model. For example, the incremental learning can be performed using the data set necessary and sufficient to output a feature point required for each medical institution or medical scene where the radiation imaging system 100 is used.

Training data used for the incremental learning may include a data set in which an optical image is input data and data or an image in which a label or the like indicating a feature point is attached to the position of the feature point in the optical image for the feature point for which the incremental learning is desired is output data (ground truth). The ground truth may be generated based on an optical image by a physician or the like. If an optical image is input, a learned model has been trained using such training data can output the coordinates of each feature point including feature point, for which the incremental learning is performed, in the input optical image, and a probability representing the likelihood of the feature point. If the probability is 0.0, the learned model may not output the position of the feature point. Training data of the general purpose learned model before performing the incremental learning may also include a data set of a similar format. However, the training data used for the incremental learning includes skeletal information related to a skeletal structure different from a skeletal structure shown by skeletal information for which the general-purpose learned model has been trained. The learned model used by the skeletal structure estimating unit 202 may have already been performed the incremental learning, and it is not necessary to perform the learning every time when the processing is performed.

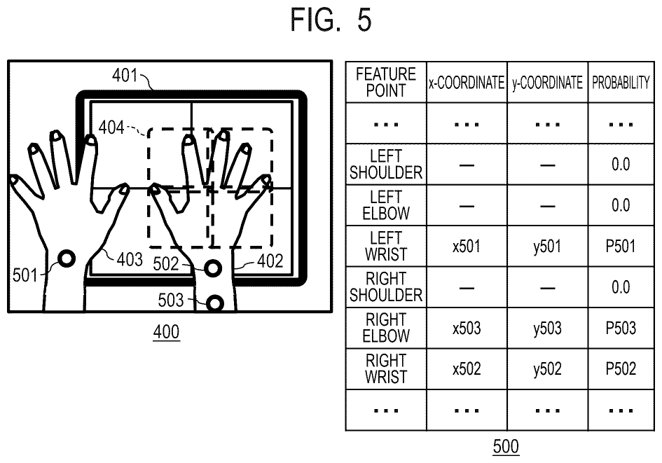

In the first example, an example will be described in which the skeletal structure estimating unit 202 applies arm skeletal structure estimation in which the left wrist, the left elbow joint, the left shoulder joint, the right wrist, the right elbow joint, and the right shoulder joint are estimated as the skeletal information, to the optical image 400. In the first example, the skeletal structure estimating unit 202 inputs the optical image 400 to the learned model. In this case, as shown in FIG. 5, it is assumed that learned model detects the left wrist 501 and the right wrist 502, and outputs the coordinates (x501, y501) and (x502, y502) and the probabilities P501 and P502 representing the likelihood of the feature points, respectively. On the other hand, it is assumed that learned model estimates the probability that the left shoulder joint, the left elbow joint, and the right shoulder joint are included in the optical image 400 as 0.0, and does not output the respective coordinates corresponding to them. On the other hand, it is assumed that learned model estimates that the right elbow joint 503 exists at the coordinate (x503, y503) with the probability P503. Here, this estimation for the right elbow joint is wrong, and in this case, it is assumed that the probability P503 is smaller than the probabilities P501 and P502.

It is assumed that the skeletal structure estimating unit 202 outputs the coordinates and probability of each feature point as the skeletal information in the format shown in a table 500. The skeletal structure estimating unit 202 may output the skeletal information in a format corresponding to the output of learned model. For example, the learned model may output the matrix data in the format shown in the table 500, and the skeletal structure estimating unit 202 may output the matrix data or output it in the format shown in the table 500. Further, for example, the learned model may output a map (heat map) in which a feature extracted from the optical image is visualized. In this case, the skeletal structure estimating unit 202 may output the map, or may output the position and probability of the feature point with the most probability for each of feature points in the map in the format shown in the table 500.

### (Step S303)

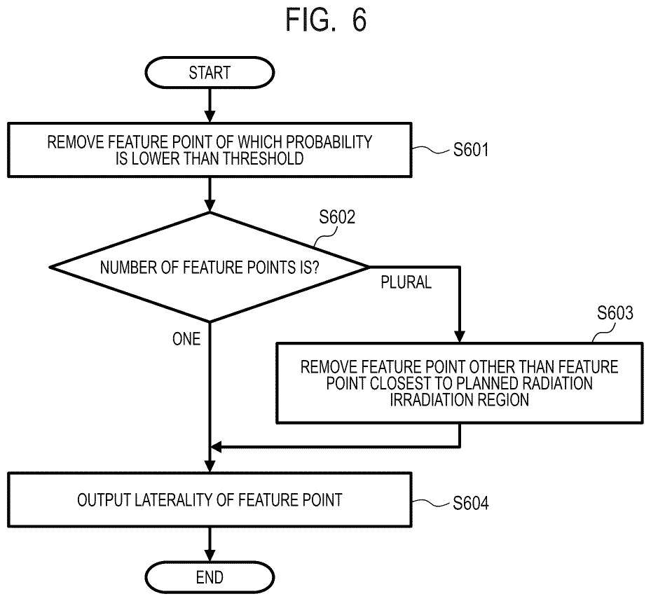

In Step S303, the object information determining unit 203 determines the laterality of the object, which is an imaging-target of the radiation image, from the skeletal information estimated by the skeletal structure estimating unit 202, and outputs the laterality as the object information. Specifically, the object information determining unit 203 applies a rule-based algorithm as shown in FIG. 6 to the skeletal information estimated by the skeletal structure estimating unit 202. In this rule-based algorithm, the laterality of the imaging-target is determined based on the probability and coordinates included in the skeletal information which is the output of the skeletal structure estimating unit 202.

Here, an example will be described in which the rule-based algorithm shown in FIG. 6 is applied to the output of the skeletal structure estimation shown in FIG. 5. The output of the skeletal structure estimation shown in FIG. 5 includes the left wrist 501, the right wrist 502, and the right elbow joint 503, and it is assumed that the probability P503 of the right elbow joint 503 is small.

First, in step S601, the object information determining unit 203 removes a feature point whose probability is less than a threshold value from among the feature points included in the skeletal information output by the skeletal structure estimating unit 202. In the above example, the object information determining unit 203 determines that the probability P503 of the right elbow joint 503 is less than the threshold value, and removes the right elbow joint 503 which is a feature point.

Next, in step S602, the object information determining unit 203 determines whether the number of remaining feature points is one or plural. If it is determined in step S602 that the number of feature points is plural, the process proceeds to step S603. On the other hand, if it is determined in step S602 that the number of feature points is one, the process proceeds to step S604. In the above example, since the object information determining unit 203 determines that the remaining feature points are two which are the left wrist 501 and the right wrist 502, the process proceeds to step S603.

In step S603, the object information determining unit 203 determines the position of a planned radiation irradiation region (irradiation region), in where the irradiation of radiation is planned, in the optical image, and removes feature points other than a feature point closest to the position of the planned radiation irradiation region among the remaining feature points. In the above example, the object information determining unit 203 calculates the distance between the planned radiation irradiation region and the coordinates (x501, y501) of the left wrist, and the distance between the planned radiation irradiation region and the coordinates (x502, y502) of the right wrist.

Here, the position of the planned radiation irradiation region in the optical image 400 may be the position of the collimator lamp irradiation region 404. Therefore, the object information determining unit 203 may extract the collimator lamp irradiation region 404 by the threshold processing based on, for example, the intensity of the optical image, etc., consider the collimator lamp irradiation region 404 as the planned radiation irradiation region, and calculate the distance between it and the feature point. The extraction method of the collimator lamp irradiation region 404 in the optical image is not limited to this, and any known method may be used. For example, the object information determining unit 203 may extract the collimator lamp irradiation region 404 by the edge detection, the corner detection, or the like.

The distance dn between the coordinates (xn, yn) of the feature point n and the representative point (for example, the center position (xc, yc)) of the planned radiation irradiation region can be determined according to the following equation 1.

$$dn = \sqrt{{( xc - xn )}^{2} + {( yc - yn )}^{2}} \qquad ( Equation 1 )$$

The object information determining unit 203 compares the distances between each of the feature points and the planned radiation irradiation region determined using the equation 1 to determine the feature point closest to the planned radiation irradiation region, and can remove feature points other than the determined feature point. In the above example, since the right wrist 502 is close to the planned radiation irradiation region among the output of the skeletal structure estimation shown in FIG. 5, the object information determining unit 203 removes the left wrist 501 which is a feature point.

In step S604, the object information determining unit 203 outputs the laterality of the remaining feature point as the object information. In the above example, the object information determining unit 203 outputs the laterality “right” of the right wrist 502 which is the remaining feature point. If the laterality of feature point is output in step S604, the determination processing of the laterality which is the determination processing of the object information according to the first example ends.

In the determination processing of the object information, the correspondence relationship between the remaining feature point and the object information to be output may be determined in advance by a rule. For example, the laterality to be output may be set to “right” if the right elbow joint or the right wrist remains as the feature point, and the laterality to be output may be set to “left” if the left elbow joint or the left wrist remains as the feature point.

The case where the feature points are the right wrist and the left wrist has been described above. However, the shoulder joint, the elbow joint, the hip joint, the knee joint, or the ankle may be used as feature point, other than them. Also in this case, the object information determining unit 203 can output the laterality of a feature point as the object information for the feature point included in the skeletal information output by the skeletal structure estimating unit 202.

The rule in the determination processing of the object information is not limited to the above rules. For example, in the determination processing of the object information, if the remaining feature point is the “right shoulder joint” or the “left shoulder joint” and the laterality to be determined is laterality related to the “hand”, some error may have occurred and therefore a rule such as outputting an error may be considered.

The planned radiation irradiation region is not limited to the collimator lamp irradiation region and may be determined using any other known method. For example, the center of the optical image may be simply set to the planned radiation irradiation region. In this case, the object information determining unit 203 may output the laterality of the feature point near the center of the optical image as the object information. In this case, the laterality of the object can be determined even if the collimator lamp irradiation region is not imaged in the optical image. Further, for example, a marker indicating the planned radiation irradiation region, or the like may be provided on an imaging table, and the planned radiation irradiation region may be determined by extracting the marker in the optical image by the object information determining unit 203.

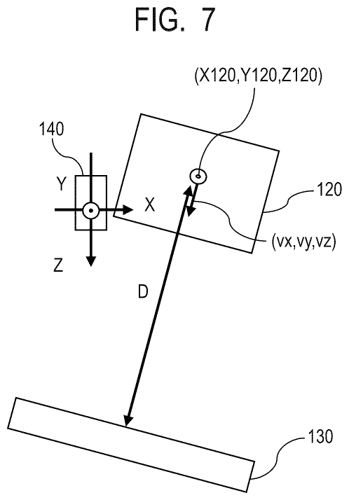

Further, if higher accuracy in the determination of the object information is desired, the planned radiation irradiation region in the image may be calculated in consideration of the spatial positions of the radiation generating apparatus 120 and the camera 140. For example, as shown in FIG. 7, it is assumed that a camera coordinate system in which the optical center of the camera 140 is the origin (0, 0, 0), the optical axis direction of the camera 140 is a Z-axis direction, and the horizontal direction and the vertical direction of the image are an X-axis direction and a Y-axis direction, respectively. Here, a case where the spatial coordinates (X120, Y120, Z120) of the radiation generating apparatus 120 and the radiation irradiation direction (vx, vy, vz) are known is assumed.

This information can be obtained, for example, by installing the camera 140 on the radiation generating apparatus 120 while measuring the position with a measuring instrument or the like. The information may also be obtained based on the amount of drive from the installation position of the radiation generating apparatus 120 or the like by using a configuration in which the amount of drive can be determined mechanically, for example, a stepping motor or the like. Further, the information may be obtained by attaching a gyro mechanism, an acceleration sensor or the like to the radiation generating apparatus 120 to obtain the displacement of position and angle from the initial position and inputting them to the information processing apparatus 200. However, in a case where the camera 140 is installed on the radiation generating apparatus 120, the spatial coordinates (X120, Y120, Z120) of the radiation generating apparatus 120 and the radiation irradiation direction (vx, vy, vz) in the camera coordinate system after the installation may generally be regarded as fixed values. In this case, the coordinates (xp, yp) of the planned radiation irradiation region in the obtained optical image can be expressed by the following equation 2, where D is the distance between the radiation generating apparatus 120 and the radiation detector 130, (fx, fy) is the focal length of the camera 140, and the coordinates (cx, cy) is the optical center.

$$xp = fx \cdot \frac{X 120 + vx \cdot D}{Z 120 + vz \cdot D} + cx , yp = fy \cdot \frac{Y 120 + vy \cdot D}{Z 120 + vz \cdot D} + cy \qquad ( Equation 2 )$$

If the planned radiation irradiation region (xp, yp) is determined as described above, the object information determining unit 203 outputs, as the object information, the laterality of the feature point which is closest to the planned radiation irradiation region (xp, yp), so that the laterality of the object can be obtained more accurately.

### (Example of Utilization of Determined Object Information)

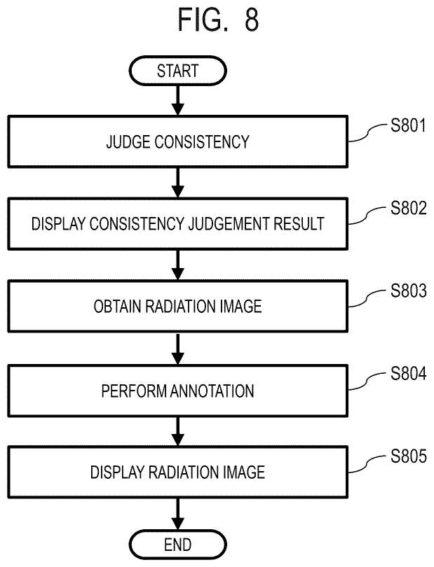

An example of the processing using the object information obtained above will be described with reference to FIG. 8. In this example, when the processing using object information is started, the object information obtained in the determination processing of the object information is transferred to the consistency judging unit 204 via, for example, the CPU bus 230, and the processing proceeds to step S801. In step S801, the consistency judging unit 204 judges the consistency between the imaging order information and the object information. For example, in a case where the imaging-target site of the imaging order is “hand” and the laterality is “right”, the consistency judging unit 204 outputs “match” if the laterality included in the obtained object information is “right”, and outputs “mismatch” otherwise.

In step S802, the display controlling unit 207 causes the display unit 235 to display the optical image obtained by the optical image obtaining unit 201, the object information output by the object information determining unit 203, the imaging order, and the result of the consistency judgement. The display controlling unit 207 can also cause the display unit 235 to display the object region and the skeletal information obtained by the skeletal structure estimating unit 202 and the analysis information obtained during the determination processing of the object information.

In step S803, after the operator confirms the information displayed on the display unit 235, the operator inputs a radiation image imaging instruction to the information processing apparatus 200 via the operation unit 234. The radiation image obtaining unit 205 obtains a radiation image in response to the radiation image imaging instruction from the operator. Specifically, the radiation image obtaining unit 205 causes the radiation generating apparatus 120 to irradiate the radiation beam with the imaging-condition corresponding to the imaging order, and obtains the radiation image from the radiation detector 130 that has detected the radiation transmitted through the object O. The radiation image obtaining unit 205 may obtain the radiation image in response to the completion of the consistency determination process. In this case, the radiation image obtaining unit 205 may obtain, for example, the radiation image corresponding to the optical image used in the determination process of the object information from the external storage apparatus 160, the storage 232, or the like.

In step S804, the object information is transferred to the annotation unit 206, for example, via the CPU bus 230, and the annotation unit 206 annotates the object information in the obtained radiation image. For example, since the radiation image is a transparent image, even if the radiation image has been obtained by imaging the right hand, it is difficult to determine whether the object is the left or right hand from the radiation image. However, the annotation unit 206 can add information that object in the radiation image is the right hand to the radiation image by arranging, for example, the information “right” as the laterality information in the radiation image as the annotation information. The radiation image on which the object information is annotated can be transferred to the display controlling unit 207 or the storage 232 via the CPU bus 230. The annotation unit 206 may annotate the object information on the optical image, and in this case, the optical image on which the object information is annotated can be transferred to the display controlling unit 207 or the storage 232 via the CPU bus 230.

In step S805, the display controlling unit 207 can cause the display unit 235 to display the radiation image on which the object information is annotated. The display controlling unit 207 may cause the display unit 235 to display the radiation image on which the object information is annotated and the optical image, side by side or by switching those images. If the display processing of the radiation image ends, the processing using the object information ends. The display unit 235 may display an optical image on which the object information is annotated as the optical image.

As described above, the radiation imaging system 100 according to the first example includes the radiation generating apparatus 120 and the radiation detector 130 for performing radiation imaging of an object, the camera 140 functioning as an example of an optical apparatus for performing the imaging of the optical image of the object, and the information processing apparatus 200. The information processing apparatus 200 includes the optical image obtaining unit 201, the skeletal structure estimating unit 202, and the object information determining unit 203. The optical image obtaining unit 201 functions as an example of an obtaining unit for obtaining an optical image obtained by imaging the object in an imaging scene of a radiation image. The skeletal structure estimating unit 202 functions as an example of an estimating unit for estimating skeletal information of the object in the obtained optical image by using the obtained optical image as input data of a second learned model, the second learned model obtained by performing incremental learning on a general-purpose learned model (first learned model) that estimates skeletal information relating to a skeletal structure of an object, the incremental learning performed by using skeletal information relating to a skeletal structure different from a skeletal structure indicated by skeletal information learned by the first learned model. The object information determining unit 203 functions as an example of a determining unit for determining object information including information on laterality of the object in the optical image using the skeletal information of the object in the optical image.

With the above configuration, the information processing apparatus 200 according to the first example can determine the laterality of the object (radiation imaging target) as the object information by using the skeletal information estimated from the optical image. Therefore, the information processing apparatus 200 can more appropriately assist the operator's determination relating to whether the laterality of the object is appropriate or not by using the determined object information. The determined object information can also be used as additional information provided to the operator together with the live image in the radiation imaging for the medical examination. Furthermore, the learned model used for estimating the skeletal information according to the first example is obtained by performing the incremental learning on a general-purpose learned model according to the purpose of each medical institution or medical scene. Therefore, the information processing apparatus 200 can appropriately estimate the skeletal information required in the medical institution or the medical scene without collecting a large amount of training data.

The object information determining unit 203 may determine the object information by rule-based processing. Therefore, the information processing apparatus 200 can determine the object information by rule-based processing, which is relatively easy to adjust, without performing the adjustment of the learned model which is difficult to change the configuration. Therefore, the information processing apparatus 200 can determine the object information in accordance with rules corresponding to the practice and knowledge in the medical institution or the medical scene.

The skeletal structure estimating unit 202 may estimate coordinates of a plurality of feature points of the object in the optical image and probability that the plurality of feature points correspond to a predefined feature point of an object as the skeletal information of the object in the optical image. In this case, the object information determining unit 203 may select a feature point of the object in the optical image by using a threshold value with respect to the probability estimated by the skeletal structure estimating unit 202, and determine the object information by using the selected feature point. By such selection of the feature point, the information processing apparatus 200 can determine the object information based on a more appropriate feature point.

Further, the object information determining unit 203 may determine a planned radiation irradiation region (irradiation region) where radiation is irradiated in the optical image. The object information determining unit 203 may selects a feature point of the object in the optical image based on positional relationship between the coordinates estimated by the skeletal structure estimating unit 202 and the irradiation region, and determine the object information by using the selected feature point. By such selection of the feature point, the information processing apparatus 200 can determine the object information based on a more appropriate feature point.

The object information determining unit 203 may determine the irradiation region based on an irradiation region of a collimator lamp in the optical image or the center of the optical image. The object information determining unit 203 may also determine the irradiation region based on arrangement of the radiation generating apparatus 120 that irradiates the radiation and the camera 140 functioning as an example of an optical apparatus that generates the optical image. In this case, by considering the arrangement of the radiation generating apparatus 120 in the camera coordinate system, it is possible to perform an appropriate analysis process according to the position of the apparatuses in each the medical scene, and the analysis accuracy of the object information determining unit 203 can be improved.

The skeletal structure estimating unit 202 may also extract an object region in the optical image and estimate the skeletal information of the object in the optical image by using the object region in the optical image as the input data of the second learned model. The skeletal structure estimating unit 202 may also classify the optical image into a class indicating that the whole body of the object appears, a class indicating that a head appears, a class indicating that a hand appears, or a class indicating that a foot appears, and select and use the second learned model corresponding to the classified class. In those cases, it can be expected that the skeletal structure estimating unit 202 can estimate more appropriate skeletal information.

The second learned model may be a learned model obtained by performing incremental learning using training data less than the number of training data of the first learned model. For example, the second learned model may be a learned model obtained by performing incremental learning on the first learned model by using skeletal information for at least one feature point of left and right elbow joints, wrists, fingers, a knee joints, ankles, toes, and the front and back of hands, and the front and back of feet.

The information processing apparatus 200 may further include a consistency judging unit 204 functioning as an example of a judging unit that determines consistency between the object information and information included in an imaging order of the radiation image. In this case, the information processing apparatus 200 can more appropriately assist the operator's determination as to whether the imaging situation such as the posture of object corresponds to the imaging order by presenting the judgment result of the consistency judging unit 204.

In this connection, the information processing apparatus 200 may further include a display controlling unit 207 that causes a display unit 235 to display at least one of the optical image, object skeletal information in the optical image, the object region in the optical image, the object information, and the judgment result of the consistency judging unit 204. If the consistency judging unit 204 outputs a judgment result indicating that the consistency is not ensured, the display controlling unit 207 may cause the display unit 235 to display a warning. In this case, the information processing apparatus 200 can present to the operator that the consistency is not matched and prompt the operator to make the imaging situation such as the posture of the object correspond to the imaging order, thereby more appropriately assisting the radiation imaging.

The information processing apparatus 200 may further include an annotation unit 206 that arranges the object information in the optical image or the radiation image as annotation information. In this case, the information processing apparatus 200 can more appropriately assist the operator's determination regarding the imaging situation such as the posture of the object by presenting the optical image or the radiation image on which the annotation information is arranged. The display controlling unit 207 may cause the display unit 235 to display the annotation information or the optical image or the radiation image on which the annotation information is arranged.

### Second Example

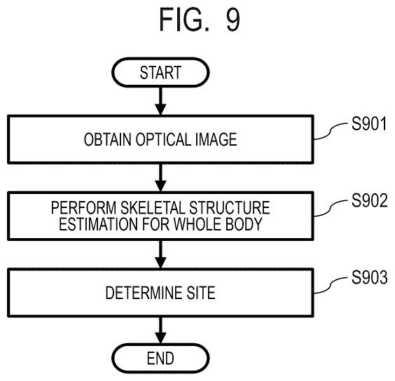

A radiation imaging system, an information processing apparatus, and an information processing method according to a second example of the present disclosure will be described below with reference to FIGS. 9 to 14. In the second example, a process of recognizing a site of an object to be imaged from an optical image obtained at a predetermined frame rate using a video camera as the camera 140 and outputting the of the object as an object information will be described. The site of object includes, for example, the head, the chest, and the limbs of a human body. Further, the granularity of the site to be recognized may be different depending on the purpose, such that, for example, the limbs can be divided into finer sites such as the upper arm, the elbow, the wrist, the hand, and the finger. As an example, a case of discriminating whether the imaging-target site of the imaging order is “chest” or “abdomen” will be described in particular.

Since the configuration of the radiation imaging system and the information processing apparatus according to the second example is the same as that of the radiation imaging system and the information processing apparatus according to the first example, the description thereof will be omitted using the same reference numerals. Further, the description of the same process as that described in detail in the first example will be omitted.

### (Process Flow)

A series of processing procedures according to the second example will be described with reference to FIG. 9. FIG. 9 is a flowchart showing a processing procedure according to the second example. When the processing procedure according to the second example is started, the process proceeds to step S901.

### (Step S901)

In step S901, the optical image obtaining unit 201 controls the camera 140 to obtain an optical image at a radiation imaging scene, the optical image including an object in radiation imaging. In the second example, the camera 140 is a video camera mounted on the radiation generator, and images the object taking an imaging position on the radiation detector 130 arranged on a recumbent table, and outputs an optical image at a predetermined frame rate.

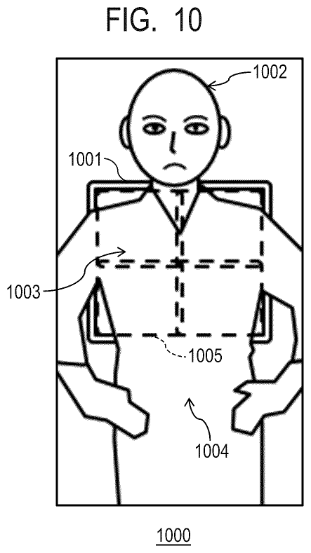

Here, an example in which the radiation detector 130, the head 1002, the chest 1003, and the abdomen 1004 appear in the optical image 1000 will be described with reference to FIG. 10. FIG. 10 is a diagram for illustrating an example of the optical image according to the second example. Here, a radiation detector region 1001 in the optical image 1000 is a region representing the radiation detector 130. Further, in the example shown in FIG. 10, it is assumed that the light of the collimator lamp is irradiated on the chest of the object, and a collimator lamp irradiation region 1005 is drawn on the chest 1003 in the optical image 1000. The collimator lamp irradiation region 1005 coincides with the planned radiation irradiation region in the radiation imaging.

### (Step S902)

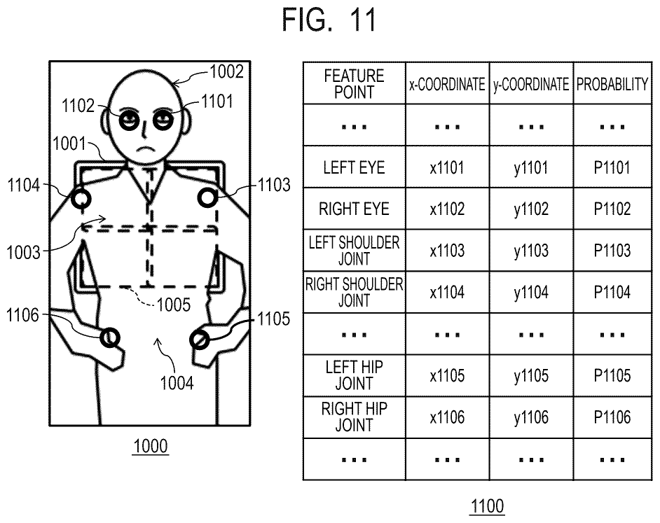

In step S902, the skeletal structure estimating unit 202 estimates skeletal information of the object by using the optical image obtained by the optical image obtaining unit 201 as input data of a learned model. In the second example, a case where the skeletal structure estimating unit 202 applies the skeletal structure estimation for whole-body to the optical image 1000, which estimates left and right eyes, left and right shoulder joints, left and right hip joints, left and right elbows, left and right wrists, left and right knees, and left and right ankles as the skeletal information will be described as an example. As shown in FIG. 11, the skeletal structure estimating unit 202 detects the left and right eyes 1101 and 1102, the left and right shoulder joints 1103 and 1104, and the left and right hip joint 1105 and 1106 in the optical image 1000 as feature points.

It is assumed that the skeletal structure estimating unit 202 outputs the coordinates and the probability of each feature point as the skeletal information in a format as shown in the table 1100. Here, it is assumed that the skeletal structure estimating unit 202 outputs the coordinates (x1101, y1101) to (x1106, y1106) of each feature point and the probabilities P1101 to P1106 indicating the likelihood of each feature point. However, it is assumed that the probability that the other skeletal information is included in the optical image 1000 is estimated as 0.0, and the coordinates corresponding to each feature point are not output. For simplicity of description, it is assumed that, unlike the example of the first example, erroneous estimation is not performed here. Note that skeletal structure estimating unit 202 may output the skeletal information in a format corresponding to the output of the learned model, similarly to the skeletal structure estimating unit 202 according to the first example.

Further, the learned model used in the second example may be a learned model that has performed the incremental learning on a learned model used for general-purpose skeletal structure estimation so that feature point required for each medical institution or medical scene can be output, similarly to the learned model according to the first example. Also, training data may be prepared similarly to the training data according to the first example.

### (Step S903)

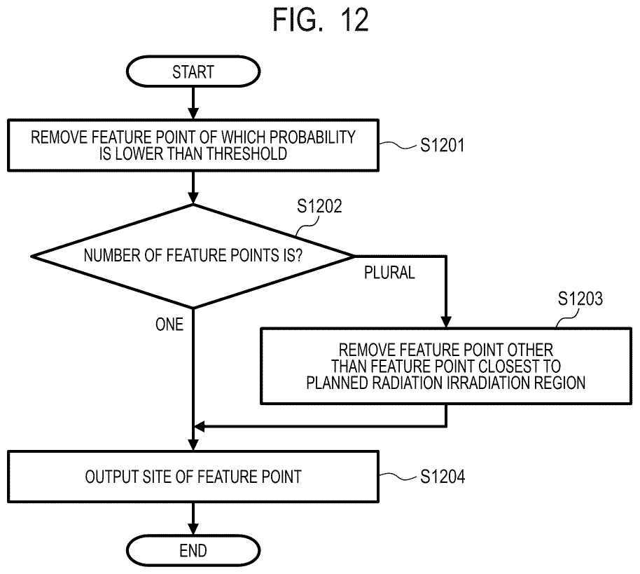

In Step S903, the object information determining unit 203 determines a site of the object, which is imaging-target of the radiation image, from the skeletal information estimated by the skeletal structure estimating unit 202, and outputs the site of the object as the object information. Specifically, the object information determining unit 203 applies a rule-based algorithm as shown in FIG. 12 to the skeletal information estimated by the skeletal structure estimating unit 202. In this rule-based algorithm, the site of the imaging-target is determined based on the probability and the coordinates of the skeletal information which is the output of the skeletal structure estimating unit 202.

As an example, a case where the rule-based algorithm shown in FIG. 12 is applied to the output of the skeletal structure estimation shown in FIG. 11 to determine whether the site of the object is the chest or the abdomen will be described. It is assumed that the output of the skeletal structure estimation shown in FIG. 11 includes the left and right eyes 1101 and 1102, the left and right shoulder joints 1103 and 1104, and the left and right hip joints 1105 and 1106, and that the probabilities P1101 to P1106 are all equal to or greater than a predetermined threshold.

First, in step S1201, the object information determining unit 203 removes a feature point whose the probability is less than the threshold value from among the feature points included in the skeletal information output by the skeletal structure estimating unit 202. In the above example, since the probabilities P1101 to P1106 are all equal to or greater than the predetermined threshold value, there is no feature point to be removed as a feature point below the threshold value.

Subsequently, in step S1202, the object information determining unit 203 determines whether the number of the remaining feature points is one or plural. If it is determined in step S1202 that the number of feature points is plural, the process proceeds to step S1203. On the other hand, if it is determined in step S1202 that there is one feature point, the process proceeds to step S1204. In the above example, since the object information determining unit 203 determines that the number of remaining feature points is plural, the process proceeds to step S1203.

In step S1203, the object information determining unit 203 obtains the position of a planned radiation irradiation region in the optical image, and remove feature points other than a feature point closest to the position of the planned radiation irradiation region among the remaining feature points. In the above example, the object information determining unit 203 calculates the distance between the position of the planned radiation irradiation region and the coordinates (x1101, y1101) to (x1106, y1106) of the feature points, respectively.

Here, the position of the planned radiation irradiation region in the optical image 1000 may be the collimator lamp irradiation region 1005. Therefore, similarly to the first example, the object information determining unit 203 may extract the collimator lamp irradiation region 1005 by the threshold processing based on, for example, the intensity in the optical image, and the like, and consider the collimator lamp irradiation region 1005 as the planned radiation irradiation region to calculate the distance between it and the feature point. The planned radiation irradiation region may be calculated in consideration of the center of the optical image or the spatial locations of the radiation generating apparatus 120 and the camera 140, as described in the first example.

The object information determining unit 203 compares the distances between each feature point and the planned radiation irradiation region, determines the feature point closest to the radiation disease region, and can removes feature points other than the feature point. In the above example, since any one of the left and right shoulder joints 1103 and 1104 is close to the planned radiation irradiation region in the output of the skeletal structure estimation shown in FIG. 11, the object information determining unit 203 removes the other feature points.

In step S1204, the object information determining unit 203 outputs the site of the remaining feature point as the object information. In the above example, the object information determining unit 203 outputs the site corresponding to any one of the left and right shoulder joints 1103 and 1104 that are the remaining feature points. Since the rule-based algorithm according to the second example is an algorithm for determining the chest or the abdomen, the “chest” close to the shoulder joint is output as the determination result. In the algorithm for determining the chest or the abdomen, for example, the “chest” is output even if the feature point closest to the planned radiation irradiation region is one of the left and right eyes. On the other hand, the “abdomen” is output if the feature point closest to the planned radiation irradiation region is one of the left and right hip joint 1105 and 1106, or if the feature point closest to the planned radiation irradiation region is one of the left and right wrists or knee joints which are not shown here. If the site of the object is output in step S1204, the determination process of the site, which is the determination process of the object information for the second example, ends.

The case where the feature points are the left and right eyes, shoulder joints, and hip joints and it is determined whether the site of the object is the chest or the abdomen has been described above. However, the process by the object information determining unit 203 for outputting the site as the object information may use other feature point, and determine other sites such as the head and limbs. The rule-based algorithm for determining the object information may be changed depending on the imaging position adopted by the medical institution or medical scene, the difference in feature points appearing in the angle of view of the camera 140 to be used, and the like. For example, in the rule-based algorithm for determining the object information including the head and limbs, the “head” may be output if the feature point closest to the planned radiation irradiation region is one of the left and right eyes. In the rule-based algorithm for determining the object information including the left and right wrists or knee joints, the site with granularity such as the “left wrist”, the “right wrist”, the “left knee joint”, and the “right knee joint” can be output.

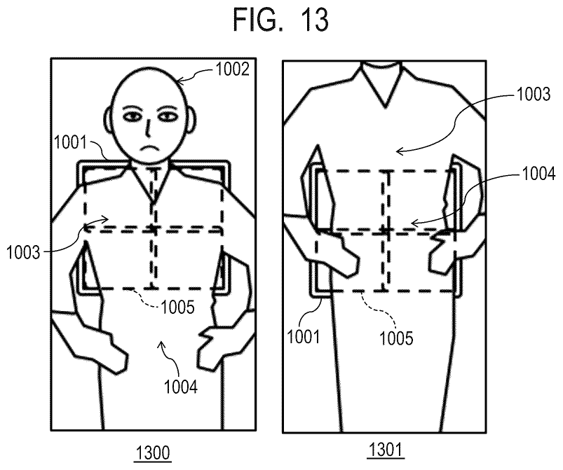

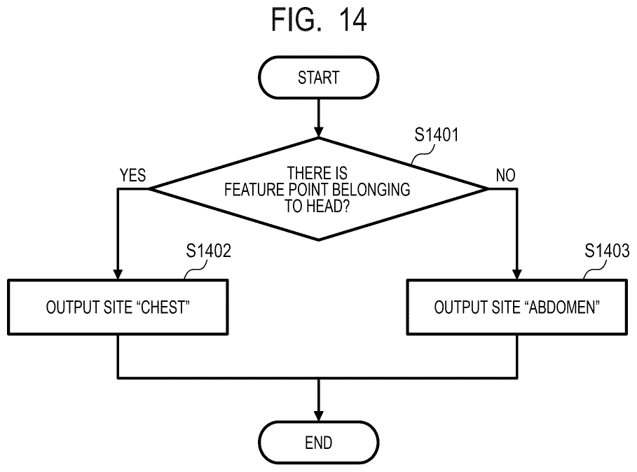

There are other rule-based algorithms for determining the site of the object. For example, as shown in FIG. 13, it is assumed that in one medical institution, the cameras 140 are arranged such that an optical image in the chest imaging is an optical image 1300 and an optical image in the abdomen imaging is an optical image 1301. In this case, the object information determining unit 203 may apply a rule-based algorithm as shown in FIG. 14 to the output of the skeletal structure estimation.

In the rule-based algorithm shown in FIG. 14, first, in step S1401, the object information determining unit 203 simply determines whether or not there is a feature point belonging to the head. If it is determined in step S1401 that there is the feature point belonging to the head, the process proceeds to step S1402. In step S1402, the object information determining unit 203 outputs the “chest” as the object information. On the other hand, if it is determined in step S1401 that there is no feature point belonging to the head, the process proceeds to step S1403. In step S1403, the object information determining unit 203 outputs the “abdomen” as the object information. If the site of the object is output in step S1402 or step S1403, the determination process of the site, which is the determination process of the object information according to the second example, ends.

As described above, the rule-based algorithm for determining the object information may correspond to the practice in a medical institution or a medical scene where the radiation imaging system 100 or the information processing apparatus 200 is used. Here, the practice in the medical institution or the medical scene may include, for example, an agreement concerning the posture of the object and the arrangement of the camera 140 at the time of the radiation imaging according to the imaged site.

### (Example of Utilization of the Determined Object Information)

The object information including the site of the object determined as described above may be used for processing such as annotation in the same manner as the first example. For example, the object information including the determined site of the object is transferred to the consistency judging unit 204 via the CPU bus 230, and may be used for consistency judgement between the imaging order information and the object information. The display controlling unit 207 can also cause the display unit 235 to display the object information and the analysis information obtained in the determination process of the object information. Furthermore, the annotation unit 206 can annotate the object information with the obtained radiation image. The display controlling unit 207 can also cause the display unit 235 to display the radiation image on which the object information is annotated.

As described above, the object information determining unit 203 can determine the object information including information indicating a site of the object in the optical image by using the skeletal information of the object in the optical image. With the above configuration, the information processing apparatus 200 according to the second example can determine the site of the object (radiation imaging target) as the object information by using the skeletal information estimated from the optical image. Therefore, the information processing apparatus 200 can more appropriately assist the operator's determination as to whether the site of the object is appropriate or not by using the determined object information. Further, as in the first example, the determined object information can be used as additional information provided to the operator together with the live image in the radiation imaging for the medical examination. Further, as in the first example, the information processing apparatus 200 can appropriately estimate the skeletal information required in a medical institution or a medical scene without collecting a large amount of training data. In the second example, the object information including information indicating the site of the object is determined, but information indicating the laterality and the site of the object may be determined as the object information.

### Third Example

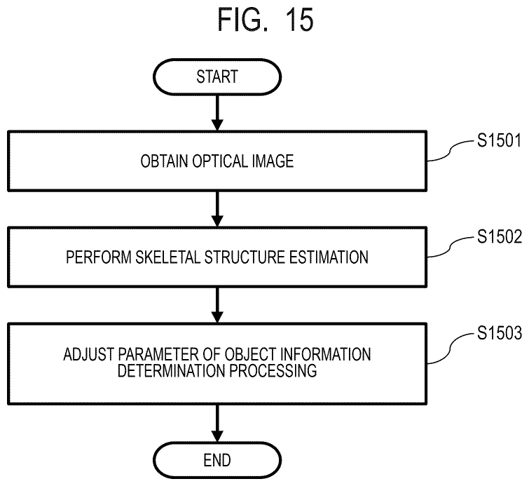

A radiation imaging system, an information processing apparatus, and an information processing method according to a third example of the present disclosure will be described below with reference to FIG. 15. In the third example, customization (adjustment) processing for recognizing laterality or a site of an object to be imaged from an optical image obtained at a predetermined frame rate by using a video camera as the camera 140 and outputting it as the object information will be described.

Since the configuration of the radiation imaging system and the information processing apparatus in the third example is the same as the configuration of the radiation imaging system and the information processing apparatus in the first example, the description thereof will be omitted using the same reference numerals. Further, the description of the same processes as those described in detail in the first and second examples will be omitted.

### (Process Flow)

Referring to FIG. 15, a process procedure of an adjustment process of a rule-based algorithm in determination process of object information according to the third example will be described. FIG. 15 is a flowchart showing the process procedure according to the third example. When the adjustment process of the rule-based algorithm in the object information determination process in the third example is started, the process proceeds to step S1501.

### (Step S1501)

In step S1501, the optical image obtaining unit 201 controls the camera 140 to obtain an optical image at an imaging scene of the radiation, the optical image including an object in radiation imaging. In the third example, the camera 140 is a video camera mounted on a radiation generator, and images the object taking an imaging posture on the radiation detector 130 arranged on a recumbent table, and outputs an optical image at a predetermined frame rate. Here, as in of the second example, a case in which the radiation detector 130, the head 1002, the chest 1003, and the abdomen 1004 appear in the optical image 1000 will be described using FIG. 10 as an example.

### (Step S1502)

In step S1502, the skeletal structure estimating unit 202 estimates skeletal information of the object by using the optical image obtained by the optical image obtaining unit 201 as input data of a learned model. In the third example, a case in which the skeletal structure estimating unit 202 applies the skeletal structure estimation for whole-body, in which the left and right eyes, the left and right shoulder joints, the left and right hip joints, the left and right elbows, the left and right wrists, the left and right knees, and the left and right ankles are estimated as the skeletal information, to the optical image 1000 will be described as an example. As shown in FIG. 11, similarly to the second example, the skeletal structure estimating unit 202 can detect the left and right eyes 1101 and 1102, the left and right shoulder joints 1103 and 1104, and the left and right hip joints 1105 and 1106 in the optical image 1000 as feature points.

A learned model used in the third example may be a learned model that has performed incremental learning on a learned model used for general-purpose skeletal structure estimation so that feature point required for each medical institution or medical scene can be output, as is the case with learned model according to the first example. Further, training data may be prepared in the same manner as the training data of the first example.

### (Step S1503)

In Step S1503, the object information determining unit 203 adjusts the parameters of the rule-based algorithm for determining a site of the object which is an imaging-target of the radiation image in response to input from the operator via the operation unit 234. More specifically, the display controlling unit 207 causes the display unit 235 to display the skeletal information estimated by the skeletal structure estimating unit 202. The operator instructs the site of the object to be output as the object information to the information processing apparatus 200 via the operation unit 234 based on the skeletal information displayed on the display unit 235. The object information determining unit 203 adjusts various parameters of the rule-based algorithm as shown in FIG. 12 in response to the instruction from the operator.

The parameters of the rule-based algorithm may include, for example, the threshold value of the threshold processing applied to the probability in step S1201, and the number of feature points used as a reference for the determination of branching the process in step S1202. The parameters of the rule-based algorithm may include the planned radiation irradiation region and a positional relationship between the feature point to be retained and the planned radiation irradiation region in step S1203, and a correspondence relationship between the feature point to be retained and the site to be output in step S1204. The positional relationship between the feature point to be retained and the planned radiation irradiation region may include, for example, the order of closeness to the planned radiation irradiation region regarding the feature point to be retained (e.g. closest or second closest, etc.).

For example, a case in which the operator inputs instruction such that the site determined from the estimated skeletal information is the “chest” via operation unit 234 in order to adjust the rule-based algorithm shown in FIG. 12 will be described. In this case, the object information determining unit 203 determines the planned radiation irradiation region, and determines whether the feature point closest to the planned radiation irradiation region is the left or right eye 1101 or 1102, the left or right shoulder joint 1103 or 1104, or the left or right hip joint 1105 or 1106. In the above example, one of the shoulder joints 1103 and 1104 is closest. Therefore, the object information determining unit 203 adjusts the parameter of the rule-based algorithm to determine and output the object information as the “chest” if the shoulder joint is closest to the planned radiation irradiation region. In this case, for example, the object information determining unit 203 may adjust the positional relationship between the feature point to be retained and the planned radiation irradiation region so that it is determined in step S1203 that one of the shoulder joints 1103 and 1104 is the feature point to be retained. The object information determining unit 203 may also adjust the correspondence relationship between the remaining feature point and the site name to be output in step S1204.

Further, the object information determining unit 203 may cause the storage 232 to store the coordinates of feature point whose probability is equal to or greater than the threshold among the skeletal information output by the skeletal structure estimating unit 202 together with the site information input by the operator without calculating the planned radiation irradiation region. In this case, information such as the coordinates of feature point in a case where the site information is registered as the chest and the coordinates of feature point in a case where the site information is registered as the abdomen are stored in the storage 232. In such a case, when a certain optical image is inputted to obtain the site of the object, the object information determining unit 203 determines the sum of the distances between the coordinates of feature point obtained by inputting the optical image and the stored coordinates of feature points, and determines the site having the minimum sum as the object information.

In the method of adjusting the parameters of the rule-based algorithm, a plurality of combinations of parameters may be prepared in advance, and any one of the combinations of parameters may be selected in response to the instruction from the operator. For example, the object information determining unit 203 may refer to a lookup table prepared in advance in response to the instruction from the operator, select a combination of parameters, and adjust the parameters. The combinations of parameters may include, for example, a combination of parameters related to the process as shown in FIG. 12, a combination of parameters related to a process using the coordinates of a feature point equal to or greater than a threshold value, and a combination of parameters related to a process as shown in FIG. 14. The combinations of parameters may include, for example, a combination of parameters related to a process for determining the laterality of the object as shown in FIG. 12. The instruction from the operator may specify the object information to be output as described above, or may specify a combination of parameters.

When the parameters of the rule-based algorithm are adjusted in step S1503, the series of processes ends.

In the third example, an example of adjusting the parameters of the rule-based algorithm for the process of determining the site of the object as the object information has been described. On the other hand, the parameters of the rule-based algorithm for the process of determining the laterality of the object as the object information as in the first example can be adjusted in the same manner as in the third example. In this case, the parameters of the rule-based algorithm may include, for example, the threshold value of the threshold processing applied to the probability in step S601, and the number of the feature points used as a reference for the determination of branching the process in step S602. Further, the parameters of the rule-based algorithm may include the planned radiation irradiation region and the positional relationship between the feature point to be retained and the planned radiation irradiation region in step S603, and the correspondence relationship between the remaining feature point and the laterality to be output in step S604. Also, the parameters of the rule-based algorithm for the process of determining the site and the laterality of the object as the object information can be adjusted in the same manner as in the third example.

As described above, the object information determining unit 203 according to the third example may adjust the parameters of the rule-based process based on the skeletal information of the object in the optical image and the object information obtained via the operation unit 234. Here, the parameters of the rule-based process may include at least one of a threshold value of threshold processing for selecting a feature point of the object in the optical image, the positional relationship between the feature point and an irradiation region to which the radiation is irradiated, and the correspondence relationship between the feature point and at least one of a site and laterality of the object. With the above configuration, the information processing apparatus 200 according to the third example can flexibly adapt the object information determination process to the practice and knowledge in a medical institution and a medical scene by adjusting the rules according to the practice and knowledge in the medical institution and the medical scene.

In the third example, the case where the optical image of the imaging scene of the radiation is obtained by the optical image obtaining unit 201 as in the first and second examples has been described. In contrast, the optical image used for the adjustment processing of the rule-based algorithm may be an optical image obtained at a virtual radiation imaging scene obtained by imaging a human phantom. That is, the object information determining unit 203 may adjust the parameters of the rule-based processing based on the skeletal information obtained by using the optical image obtained by imaging the phantom and the object information obtained via the operation unit 234. In this case, it is possible to obtain an image by which the skeletal structure estimation in step S1502 is performed easily than using an actual object, and more appropriate parameter adjustment can be performed in step S1503.

The optical image used for the adjustment processing of the rule-based algorithm may not be an image obtained by actual imaging. For example, a two-dimensional image obtained by projecting three-dimensional coordinates representing a virtual skeletal structure position calculated from virtual object data obtained using a three-dimensional modeling tool may be used. In this case, the object information determining unit 203 may use skeletal information output by the skeletal structure estimating unit 202 based on the two-dimensional image, for the parameter adjustment processing. That is, the object information determining unit 203 may adjust the parameters of the rule-based processing based on the skeletal information obtained by projecting the three-dimensional coordinates of the virtual object data generated by the three-dimensional modeling tool on the two-dimensional image coordinates and the object information obtained via the operation unit 234. According to this method, an image showing skeletal position in a desired posture can be relatively easily obtained on the information processing apparatus 200 without placing the human body or the phantom in the desired posture. Such a two-dimensional image may be used as input data of training data relating to the incremental learning of the learned model used by the skeletal structure estimating unit 202.

In the third example, the object information determining unit 203 adjusts the parameters of the rule-based algorithm relating to the decision processing of the object information according to the instruction from the operator. On the other hand, the information processing apparatus 200 may be provided with a programming environment, and the operator may write the handling of the skeletal information output by the skeletal structure estimating unit 202 as a program via the operation unit 234. Such a configuration may use, for example, a programming language such as C language or Python. The configuration of the information processing apparatus 200 may use a no-code development platform capable of constructing a rule-based algorithm using a graphical user interface. Furthermore, in recent years, it has become possible to use an artificial intelligence chatbot, which is a kind of generative Al, to generate a program by inputting instruction in a natural language. Therefore, the configuration of the information processing apparatus 200 may use a generative Al such as an artificial intelligence chatbot.

Further, the training data of the learned model is not limited to data obtained by using the camera 140 itself which actually performs imaging, but may be data obtained by using the same model of camera, data obtained by using the same type of camera, or the like according to a desired configuration. It is considered that the learned model for the skeletal structure estimation according to the above embodiment and examples extract, for example, the magnitude of the intensity value of the optical image, and the order, the slope, the position, the distribution, and the continuity of the bright part and the dark part, and the like as part of feature and used for the estimation processing of the skeletal information.

The learned model for the skeletal structure estimation described above may be provided in the information processing apparatus 200. An inference unit (learned model) may be configured by a software module executed by a processor such as a CPU, an MPU, a GPU, an FPGA, or the like, or may be configured by a circuit that serves a specific function such as an ASIC. The inference unit may be provided in a different apparatus such as a server connected to the information processing apparatus 200. In this case, the information processing apparatus 200 can use the inference unit by connecting to the server or the like that includes the inference unit through any network such as the Internet. The server that includes the inference unit may be, for example, a cloud server, a FOG server, an edge server, or the like. Note that, in a case where a network within the facility, or within premises in which the facility is included, or within an area in which a plurality of facilities is included or the like is configured to enable wireless communication, for example, the reliability of the network may be improved by configuring the network to use radio waves in a dedicated wavelength band allocated to only the facility, the premises, or the area or the like. Further, the network may be configured by wireless communication that is capable of high speed, large capacity, low delay, and many simultaneous connections.

Although the present disclosure has been described above with reference to the embodiment and examples, the present invention is not limited to the above embodiments and example. The present disclosure includes the invention modified within the extent not contrary to the spirit of the present invention and the invention equivalent to the present invention. Further, the above-described embodiment and each example can be suitably combined within the extent not contrary to the spirit of the present disclosure.

According to the embodiment and the first to third examples of the present disclosure, object information can be obtained from an optical image using a learned model that estimates desired skeletal information, which is obtained using a reduced number of training data as compared to the conventional one.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The processor or circuit may include a central processing unit (CPU), a microprocessing unit (MPU), a graphics processing unit (GPU), an application specific integrated circuit (ASIC), or a field programmable gateway (FPGA). The processor or circuit may include a digital signal processor (DSP), a data flow processor (DFP), or a neural processing unit (NPU).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-182678, filed Oct. 24, 2023, which is hereby incorporated by reference herein in its entirety.

What is claimed is:

1. An information processing apparatus comprising:
an obtaining unit configured to obtain an optical image obtained by imaging an object in an imaging scene of a radiation image;
an estimating unit configured to estimate skeletal information of the object in the obtained optical image by using the obtained optical image as input data of a second learned model, the second learned model obtained by performing incremental learning on a first learned model that estimates skeletal information relating to a skeletal structure of an object, the incremental learning performed by using skeletal information relating to a skeletal structure different from a skeletal structure indicated by skeletal information learned by the first learned model; and
a determining unit configured to determine object information which includes information of at least one of laterality and a site of the object in the optical image by using the skeletal information of the object in the optical image.

2. The information processing apparatus according to claim 1, wherein the determining unit is configured to determine the object information by rule-based processing.

3. The information processing apparatus according to claim 1, wherein the estimating unit is configured to estimate coordinates of a plurality of feature points of the object in the optical image and probability that the plurality of feature points correspond to a predefined feature point of an object as the skeletal information of the object in the optical image.

4. The information processing apparatus according to claim 3, wherein the determining unit is configured to select a feature point of the object in the optical image by using a threshold value with respect to the probability estimated by the estimating unit, and determine the object information by using the selected feature point.

5. The information processing apparatus according to claim 3, wherein the determining unit is configured to:
determine an irradiation region to which radiation is irradiated in the optical image; and
select a feature point of the object in the optical image based on positional relationship between the coordinates estimated by the estimating unit and the irradiation region; and
determine the object information by using the selected feature point.

6. The information processing apparatus according to claim 5, wherein the determining unit is configured to determine the irradiation region based on an irradiation region of a collimator lamp in the optical image or the center of the optical image.

7. The information processing apparatus according to claim 5, wherein the determining unit is configured to determines the irradiation region based on arrangement of a radiation generating apparatus that irradiates the radiation and an optical apparatus that generates the optical image.

8. The information processing apparatus according to claim 1, wherein the estimating unit is configured to extract an object region in the optical image, and estimate the skeletal information of the object in the optical image by using the object region in the optical image as the input data of the second learned model.

9. The information processing apparatus according to claim 1, wherein the estimating unit is configured to classify the optical image into a class indicating that a whole body of the object appears, a class indicating that a head appears, a class indicating that a hand appears, or a class indicating that a foot appears, and select and use the second learned model corresponding to the classified class.

10. The information processing apparatus according to claim 1, wherein the second learned model is a learned model obtained by performing incremental learning using training data less than the number of training data of the first learned model.

11. The information processing apparatus according to claim 1, wherein the second learned model is a learned model obtained by performing incremental learning on the first learned model by using skeletal information for at least one feature point of left and right elbow joints, wrists, fingers, knee joints, ankles, toes, and the front and back of hand, and the front and back of feet.

12. The information processing apparatus according to claim 1, further comprising a judging unit configured to determine the consistent between the object information and information included in an imaging order of the radiation image.

13. The information processing apparatus according to claim 12, further comprising a display controlling unit configured to cause a display unit to display at least one of the optical image, the skeletal information of the object in the optical image, an object region in the optical image, the object information, and the judgement result by the judging unit.

14. The information processing apparatus according to claim 12, further comprising a display controlling unit configured to cause a display unit to display a warning if the judging unit outputs a judgement result indicating that the consistency is not ensured.

15. The information processing apparatus according to claim 1, further comprising an annotation unit configured to arrange the object information in the optical image or the radiation image as annotation information.

16. The information processing apparatus of claim 15, further comprising a display controlling unit configured to cause a display unit to display at least one of the optical image, the skeletal information of the object in the optical image, an object region in the optical image, the object information, the annotation information, and an optical image or a radiation image in which the annotation information is arranged.

17. The information processing apparatus according to claim 2, wherein the determining unit is configured to adjust parameters of the rule-based processing based on the skeletal information of the object in the optical image, skeletal information obtained by using an optical image obtained by imaging a phantom, or skeletal information obtained by projecting three-dimensional coordinates of virtual object data generated by a three-dimensional modeling tool on two-dimensional image coordinates, and object information obtained via an operation unit.

18. A radiation imaging system comprising:
an optical apparatus arranged to imaging an optical image of an object;
a radiation detector arranged to perform radiation imaging of the object; and
the information processing apparatus according to claim 1.

19. An information processing method comprising:
obtaining an optical image obtained by imaging an object in an imaging scene of a radiation image;
estimating skeletal information of the object in the obtained optical image by using the obtained optical image as input data of a second learned model, the second learned model obtained by performing incremental learning on a first learned model that estimates skeletal information relating to a skeletal structure of an object, the incremental learning performed by using skeletal information relating to a skeletal structure different from a skeletal structure indicated by skeletal information learned by the first learned model; and
determining object information which includes information of at least one of laterality and a site of the object in the optical image by using the skeletal information of the object in the optical image.

20. A non-transitory computer-readable storage medium having stored thereon a program that, when executed by a computer, causes the computer to execute the information processing method according to claim 19.
